(19) United States
(12) Patent Application Publication
(10) Pub. No.: US 2025/0160627 A1
(43) Pub. Date: May 22, 2025

(54) METHODS, APPARATUS AND SYSTEMS FOR CONTROLLING A MEDICAL DEVICE

(71) Applicants: Canon U.S.A., Inc., Melville, NY (US); CANON KABUSHIKI KAISHA, Tokyo (JP)

(72) Inventors: HuaLei Shelley Zhang, Brookline, MA (US); Takahisa Kato, Brookline, MA (US); Fumitaro Masaki, Brookline, MA (US); Brian Ninni, Woburn, MA (US); Kiyoshi Takagi, Tokyo (JP)

(21) Appl. No.: 18/834,993

(22) PCT Filed: Jan. 31, 2023
(86) PCT No.: PCT/US23/61701

§ 371 Date: Jul. 31, 2024

Related U.S. Application Data
(60) Provisional application No. 63/305,478, filed on Feb. 1, 2022.

(51) Int. Cl. A61B 1/00; A61B 1/005

(52) U.S. Cl. CPC A61B 1/0016; A61B 1/0052; A61B 1/0057

(57) ABSTRACT

A continuum robot having independently manipulateable bendable section for advancing the robot through a passage, without contacting fragile elements within the passage, wherein the robot incorporates a system, method and apparatus for transitioning between modes for ease of use by a physician, as well as a transitional park mode for ensuring correct manipulation of the continuum robot.

### CROSS REFERENCE TO RELATED PATENT APPLICATIONS

This application claims priority from U.S. Provisional Patent Application No. 63/305,478, filed on Feb. 1, 2022, in the United States Patent and Trademark Office, the disclosure of which is incorporated by reference herein, in its entirety.

### FIELD OF THE DISCLOSURE

The present disclosure relates generally to medical devices and, more particularly to a continuum robot (also referred to as ‘snake’ or ‘snake system’) applicable to guide interventional tools and instruments, such as endoscopes and catheters, in medical procedures.

### BACKGROUND OF THE DISCLOSURE

A continuum robot or snake includes a plurality of bending sections having a flexible structure, wherein the shape of the continuum robot is controlled by deforming the bending sections. The snake mainly has two advantages over an existing robot having rigid links. The first advantage is that the snake can move along a curve in a narrow space or in an environment with scattered objects, in which the rigid link robot may get stuck. The second advantage is that it is possible to operate the snake without damaging surrounding fragile elements because the snake has intrinsic flexibility.

In recent years, minimally invasive medical care, with which burden on the patient can be reduced and the quality of life (QOL) after the treatment or inspection can be improved, has been attracting attention. Surgery or inspection using an endoscope is a typical example of minimally invasive medical care. For example, a laparoscopic surgery is advantageous over a conventional abdominal surgery in that it can be performed with a smaller surgical wound, which results in a shorter stay in the hospital and less damage to the appearance.

Endoscopes used for the minimally invasive medical care are roughly divided into rigid endoscopes and soft endoscopes. With a rigid endoscope, although clear images can be obtained, the direction in which an observation target can be observed is limited. In addition, when the rigid endoscope is inserted into a curved organ, such as the esophagus, large intestine, or urethra, an insertion portion of the rigid endoscope presses the organ and causes pain for the patient. In contrast, a soft endoscope includes an insertion portion formed of a bendable member, so that a large area can be observed in detail by adjusting the bending angle of the distal end of the endoscope. In addition, by bending the insertion portion along an insertion path, burden on the patient can be reduced.

When the number of bendable portions is increased, the endoscope can be inserted to a deep area of the body without causing the endoscope to come into contact with tissue even when the insertion path has a complex curved shape.

Accordingly, soft endoscopes having a plurality of bendable portions have been widely researched and developed.

The inspection and surgery using an endoscope have a problem that operation of the endoscope requires skill. One reason for this is that the physician cannot directly observe the position of the insertion portion of the endoscope, and the relationship between the operating direction and the direction of movement in the observed image cannot be easily recognized. In particular, when the number of bendable portions is increased, the position of the insertion portion varies in a complex manner, and therefore the difficulty of operation is further increased. As a result, the time required for the inspection or surgery is increased, and burden on the physician and the patient is increased accordingly.

Various related art disclosures in the field include: WO2020243285A1 (hereafter “related art 1’), which discloses methods for a steerable medical instrument to control drive forces applied to the control wire under a passively controlled mode, where an amount of strain or an amount of displacement of the control wire is reduced to make the control wire compliant to external forces; as well as US20180192854 (hereafter “related art 2’) which provides a method and an apparatus for controlling a flexible manipulator including a plurality of bendable mechanisms via a control apparatus. The control apparatus can select among movement modes of bending movement, angled view movement, or a remote center movement; and finally US20180243900 (hereafter “related art 3’), which discloses the mechanism of leader following control (aka follow-the-leader), where the distal section is bent via a user command, the middle section is advanced in a predetermined distance, and the proximal section is bent automatically based on the bending angle of the distal portion.

However, none of the known disclosures address complications arising from robotic catheters which include many degrees of freedom necessary to control and complete the intended different tasks for the application (related art 1, 2, and 3). It would be too complex and counterintuitive for a human operator to directly command these many degrees of freedom in the robotic catheter. Motion modes, which have been seen in related art 2 and 3, can give the human operator reduced degrees of freedom to control with a unique motion rule with these many degrees of freedom in the catheter. However, when the robotic catheter system includes multiple motion modes, it becomes very complex and counterintuitive for the operator to select an appropriate motion mode among multiple motion modes for the different situations with the conventional motion switch button.

As such, the subject innovation introduces a solution to this quandary.

### SUMMARY

Thus, to address such exemplary needs in the industry, the presently disclosed apparatus teaches a robotic apparatus comprising: a continuum robot including a first bending section which is bent by at least a first wire; a driver that drives the wire; a base affixed to the driver and capable of moving the continuum robot in one axis; and an operational console that controls a movement of the driver and a movement of the base, wherein, the operational console allocates a different motion mode for the robotic apparatus based on an advancing motion or a retracting motion of the robotic apparatus.

Arobotic apparatus comprising a continuum robot including a first bending section which is bent by at least a first wire; a driver that drives the wire; a base affixed to the driver and capable of moving the continuum robot in one axis; and an operational console that controls a movement of the driver and a movement of the base, based on an input, wherein, the operational console allocates a different motion mode for the robotic apparatus based on an advancing motion or a retracting motion of the robotic apparatus; wherein the motion modes are mappings between the input and the movement of the driver and movement of the base.

In additional embodiments, the continuum robot further comprises a second bending section proximal to the first bending section, which is bent by at least a second wire driven by the driver.

Furthermore the subject innovation includes a comprising a park mode that suspends the operational console from moving the base. Wherein park mode may be initiated once the robotic apparatus is static.

In yet another embodiment, the robotic apparatus is static when there is no command for movement from the operational console.

In further embodiment, park mode is used as a transition from any other modes, including but limited to: a FTL mode, a rFTL mode, a Target mode and a BKD mode. Furthermore, the FTL mode is utilized for the advancing motion and the rFTL mode is utilized for the retracting motion, with park mode utilized between the FTL mode and the rFTL mode.

It is further contemplated that the subject innovation comprises an insertion sensor in communication with the continuum robot, to detect the advancing motion, retracting motion or static motion of the continuum robot in a cavity. In some embodiments, this insertion sensor is located on the base.

In yet another embodiment, the innovation comprises a pause mode that suspends the operational console from moving the base and the at least first wire.

The subject innovation also teaches a method for controlling a robotic apparatus: the robotic apparatus comprising: a continuum robot having a first bending section which is bent by at least a first wire; a driver that drives the wire; a base affixed to the driver and capable of moving the continuum robot in one axis; and an operational console that controls a movement of the driver and a movement of the base, wherein the method comprises: manipulating the operational console to send a signal to the driver; advancing or retracting the base using the driver based on signals from the operational console; advancing or retracting the robotic apparatus using the driver based on signals from the operational console; wherein, the operational console allocates a different motion mode for the robotic apparatus, according to an advancing motion or a retracting motion of the continuum robot.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present innovation will become apparent from the following detailed description when taken in conjunction with the accompanying figures showing illustrative embodiments of the present innovation.

FIG. 1 is a block diagram of an exemplary bendable medical device incorporating various ancillary components, according to one or more embodiment of the subject apparatus, method or system.

FIG. 2 illustrates a kinematic model of the subject continuum robot, according to one or more embodiment of the subject apparatus, method or system.

FIG. 3 provides a detailed illustration of the subject continuum robot, according to one or more embodiment of the subject apparatus, method or system.

FIG. 4 is an image and close-up of a target area in the lungs, in connection with a pathway of the subject continuum robot, according to one or more embodiment of the subject apparatus, method or system.

FIG. 5 is a diagram of various driving and park modes according to one or more embodiment of the subject apparatus, method or system.

FIG. 6 depicts a diagram of various driving, park, and target modes according to one or more embodiment of the subject apparatus, method or system.

FIG. 7 provides a diagram of various driving, park, and target modes according to one or more embodiment of the subject apparatus, method or system.

FIG. 8 shows us a diagram of various driving and target modes at different locations in the lungs, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

In the subject disclosure, Applicant will first detail the mechanism of a continuum robot, followed by the catheter attachment/detachment functionality methods, as well as the systems and procedures associated with the continuum robot and said functionality.

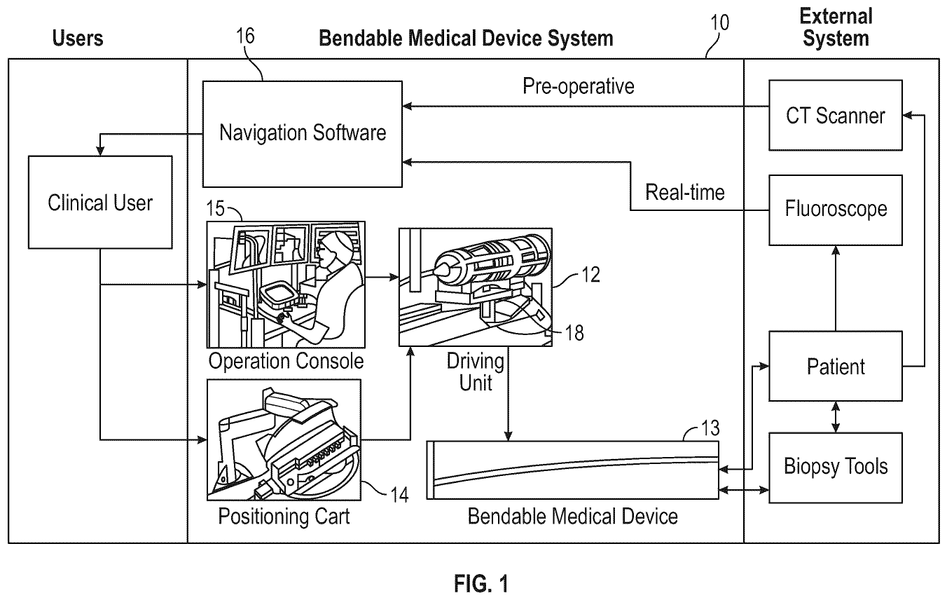

FIG. 1 is a system block diagram of an exemplary bendable medical device system 10 incorporating various ancillary components intended to amass a complete medical system. The bendable medical device system 10 comprises an actuator or driving unit 12 (also referred to herein as a ‘driver’) for driving the wires, and having a base stage 18, a bendable medical device 13, a positioning cart 14, an operation console 15, having push-button, thumbstick, and/or joystick operational console 15, and navigation software 16. The operation console 15 includes a user input device such as a push-button, thumbstick, and/or joystick. The operation console 15 also includes a processor for processing input from the user as well as input from sensors, etc. The processor further processes mapping between the input and the movement of the driver and movement of the base.

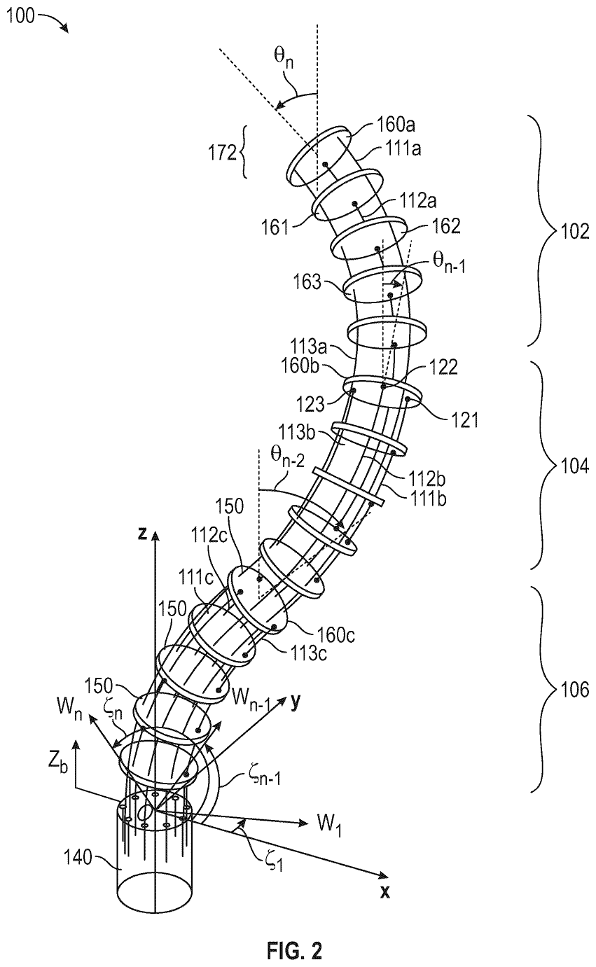

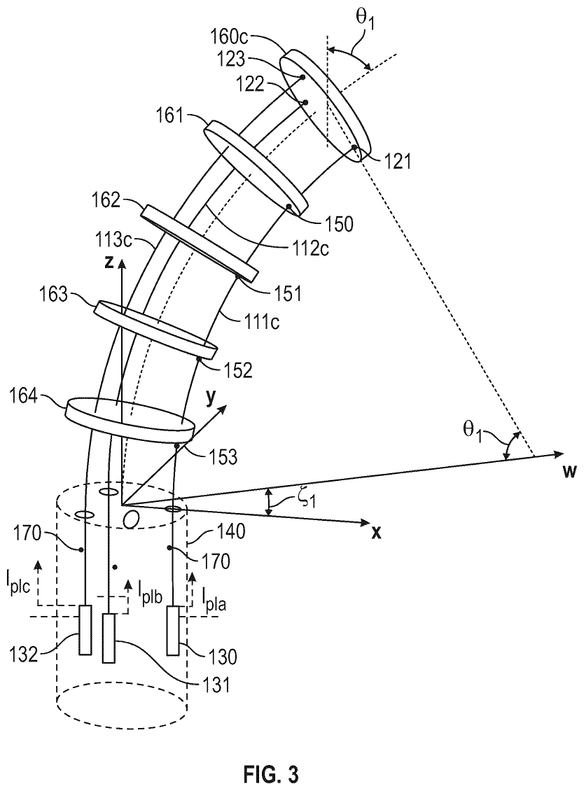

FIG. 2 illustrates a continuum robot 100 that is capable of a plurality of bends, with FIG. 3 providing an enlarged view of the proximal end of the robot 100.

The exemplary bendable medical device system 10 is capable of interacting with external system component and clinical users to facilitate use in a patient.

As shown in FIG. 2, the continuum robot 100, comprises wires 111b, 112b and 113b, which are connected to connection portions 121, 122 and 123, respectively, found on an end disc 160b, for controlling the middle bending section 104. Additional wires (3 for each of the other bendable sections 102 and 106) 111a, 111c, 112a, 112c, 113a, 113c, are attached at the distal ends of each bendable section 102 and 106, to the respective end disc 160a and 160c.

As each bending section is operated similarly, we will focus on one bending section, here the middle bending section 104, to explain the mechanism. The posture of the bending section 104 is controlled by pushing and pulling the wires 111b to 113b by using actuators 130 to 132 disposed in a robot base 140. (Note—In the interest of clarity, only actuators for the three wires 111c, 112c, 113c have been show in FIG. 3, additional actuators for the remaining 6 wires are contemplated in this innovation.)

Moreover, the robot base 140 of the continuum robot 100 is disposed on a base stage 18 (See FIG. 1) and can be moved by the base stage 18 in the longitudinal direction. Thus, it is possible to advance and retard the robot 100 into a target structure by advancing and retarding the base stage 18.

An operational console 15 (see FIG. 1) indicates a driving amount to the base stage 18 and, independently, to the actuators 130 to 132. Throughout this disclosure, the operational console 15 may also be described or eluded to as a control system or controller. The operational console 15 may include dedicated hardware including a field-programmable gate array (“FPGA”) and the like; or may be a computer including a storage unit, a work memory, and a central processing unit (“CPU”). In the case where the operational console 15 is a computer, the storage unit may store a software program corresponding to an algorithm of the control system (described below) and the central processing unit expands the program in the work memory, executes the program line by line, and thereby the computer functions as the operational console 15. In either case, the operational console 15 is communicably connected to the base stage 18 and the actuators 130 to 132, and the operational console 15 send signals representing the driving amount and configuration to these control targets, which are imputed by an end user through push buttons, joystick or the like.

The continuum robot 100 includes multiple wire guides 161 to 164 situated throughout each bending section, and moreover detailed in FIG. 3 for proximal bending section 106. The wire guides 161 to 164 are shown here guiding the wires 111c, 112c and 113c, and for providing structural integrity to the bending section 106. As before and for the sake of redundancy, we have elected to detail the components of the proximal bending section 106 in FIG. 3, with the understanding that the remaining bending sections 102 and 104, function in a similar fashion with similar elements. The wire guides 161 to 164 each contain a wire through 150-153 for each wire 111c-113c. For ease of illustration, FIG. 3 only depicts the wire through 150-153 for a single wire 111c. Alternatively, a method of discretely arranging the plurality of wire guides, a continuum robot 100 having a bellows-like shape or a mesh-like shape may be utilized, wherein the wire guides 161-164 are fixed to their respective wires 111a-113a.

With respect to FIGS. 2 and 3, the definitions of symbols are as follows: I_{d}=the length of the central axis a bending section; θₙ=the bending angle of the distal end; ζₙ=the rotational angle of the distal end; ρₙ=the radius of curvature of a bending section.

### Embodiment 1

As detailed above, the robotic catheter 100 in this embodiment includes at least one distal bending section 102 with robotic insertion and removal of the catheter 100 from the target. This embodiment will map the different motion modes as the active operation mode for the end user according to the insertion and removal motion commands without the conventional motion switch buttons.

### Example Design 1—Current Design for Lung Application

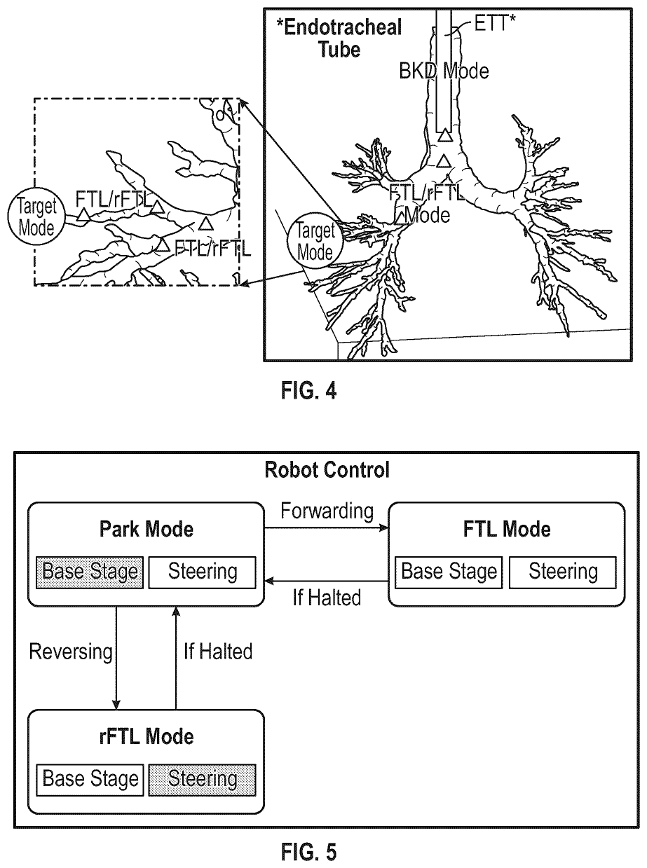

Specifically, in the first example design, the robotic catheter 100 includes three bending sections 102 (distal), 104 (middle) and 106 (proximal), and would be inserted into the lung airway. In the workflow, there is a target lesion in the lung for the robotic catheter 100 to reach (FIG. 4). The robotic catheter 100 will be navigated to the target lesion through the airways and will provide the pathway (a tool channel) to deliver appropriate tools, like biopsy tools or therapeutic tools.

For this catheter 100 navigation to the lesion, the end user will insert and remove the robotic catheter 100. During the insertion and removal, the operator will need different motion modes to steer the distal bending sections as follows: Insertion—where the operator needs to steer the distal bending section 102 to turn through the airways and select bifurcations with minimal interaction with the airways by synchronizing the insertion motion; and Removal—where the operator needs to retreat the catheter 100 along the insertion pathways by synchronizing the removal motion.

The following motion modes would reflect these needs. Insertion—Follow-the-leader (FTL) mode allows the operator to control the orientation/trajectory of the distal bending section 102, which is the leader section in this mode, by using a joystick on the operation console 15. In this operation console 15, the system would calculate the commands to the driving unit 12 based on the inputs from the joystick by using a robot kinematic and servo control between the operational console 15 and the driving unit 12 with the encoders. When the operator inserts the catheter 100 with the base stage 18, and driving unit 15, the system uses the commands for the leader section to control the following middle bending section 104 and proximal bending section 106.

Removal: Utilizing reverse follow-the-leader (rFTL) mode, the system would remember the bending commands for all three bending sections 102, 104 and 106, during the insertion mode, and when prompted by the operator for removal of the catheter through the operational console 15, the system would reversely execute the follow-the-leader mode. At this time, the operator would control only the insertion depth of the base stage 18 with the operational console 15. Therefore, we can simplify the operator's control for catheter 100 removal while minimizing interaction to the airways.

Park Mode is entered when the base is stopped, and can be entered automatically when the base is stopped. In this mode, the operator would control only the tip section. In some embodiments, the middle and proximal section of the 3-seciton robot are not moveable by the joystick. The operator can exit park mode by initiation movement of the base stage 18, in either a forward or reverse direction. An advantage of park mode is that the user can stop during an insertion or retraction to decide what direction or trajectory to follow. Another advantage of park mode is that it simplifies the transition between FTL and rFTL modes. Without park mode, the system needs to determine the previous state and switch appropriately. In rFTL, if the base motion is stopped, and the rFTL mode is maintained, the user cannot provide any control of the tip, and such control is important for, for example, the user to view various direction within the lumen.

To activate these modes, the system in this embodiment will use the insertion and removal command from the operator instead of the dedicated motion switch button. As seen in the FIG. 5, the clear boxes signify a command operable by the operator while the shaded boxes signifies commands prohibited from the operator. The system includes two buttons for the insertion and removal of the catheter. The operator can command forward or backward motion from the operational console 15 which instructs the base stage 18 and can move the catheter 100 forward when the operator is pressing the insertion button, and can move the catheter 100 backward (reversing) when the operator is pressing the removal button.

When the operator is not pressing either insertion or removal button, the catheter 100 will be idle for the insertion position (park mode, FIG. 5). The system will activate the FTL mode only during engagement of the insertion button as well as the rFTL mode only during engagement of the removal button.

### Example Design 2

By mapping the different motion modes based on the insertion and removal command, the system can provide intuitive control to the operator without involving additional operator's judgement for the appropriate motion modes for the workflow. This will also reduce human factor error in possibly selecting the wrong motion mode.

Since the catheter 100 procedure generally involves the insertion and removal motion and requires different needs for the catheter motion between the insertion and removal motions, the system mapping the different motion modes, according to the insertion and removal commands, would be beneficial with the different combination of the motions from the above-mentioned FTL and rFTL motion modes.

An example design would include the following different needs in the insertion and removal steps. Insertion: the operator needs to have relatively rigid bending sections to create a stable base for the bending section; Removal: the operator needs to have relatively flexible bending sections to remove the catheter body along with the inserted hole with minimal interaction.

A back-drivable (BKD) mode can be utilized to achieve any change in the flexibility of the bending sections. In this exemplary design [2], the actuator unit 12 and actuators 130-132, for bending the bending sections 102, 104 and 106, include at least one force sensors 170 to measure the forces on the driving wires 111(a-c)-113(a-c) in the catheter 100, with feedback force measurements by force sensors 170 in the actuation units. Those force sensors 170 (seen in FIG. 3) are integrated just after the catheter-to-actuator connector and can measure the force on the wire guides 160-164. The feedback control loop is formed with those force sensors 170 and the actuators 130-132. In this mode, the actuators 130-132 would react to the external forces to the catheter 100 to reduce the force on the wire guides 160-164 and make the catheter 100 virtually flexible. The sensors 170 are enacted to detect a status of the driving wire, wherein the status may be a force or a position relayed back to the operational console 15.

Overall, the key aspect of this innovation is to map the different motion modes according to the insertion and removal commands.

### Embodiment 2

The robotic catheter 100 in this 2^{nd} embodiment includes the two motion modes for the insertion and removal steps in Embodiment 1, and further provides the intuitive mode selection method for these motion modes.

As shown in Embodiment 1, the robotic catheter 100 including the insertion and removal motions has unique needs during inserting and removing the catheter. Besides the unique needs in the insertion and removal steps, these needs are exclusive in the insertion and removal steps. The operator should not activate any other modes except for the intended motion modes for the insertion and removal steps. With this consideration, the system in this embodiment allows the operator to activate the other motion modes when the catheter 100 is halted (stable) in terms of the insertion and removal motions, which we have come to reference herein as park mode.

The park mode is proposed to be activated whenever the base stage 18 is inactivated from moving, and to serve for all driving modes to switch over to one another.

### Example Design 1—Current Design for Lung Application

This example design is for the same lung application as in example design 1 in embodiment 1, however the system newly includes a targeting mode besides FTL and rFTL modes, where the base stage 18 is prohibited from moving, while the operator can command the distal bending section 102 of the robot while the control algorithm commands the bending angles for the middle bending section 104 and proximal bending section 106.

In this targeting mode, the system will provide fine adjustment for tip 172 translational position and orientation. The system disables the insertion and removal motion and provides joystick control of the distal bending section 102 for tip 172 translational position and tip 172 orientation. One design embodiment may provide for two thumbsticks in the joystick for controlling tip 172 translational position and orientation, separately. While the operator manipulates one of the thumbsticks to change the tip 172 translational position, the system controls the distal bending section 106 and middle bending section 104 at the same time based on the robot kinematic calculation and the servo control with the actuators 130-132.

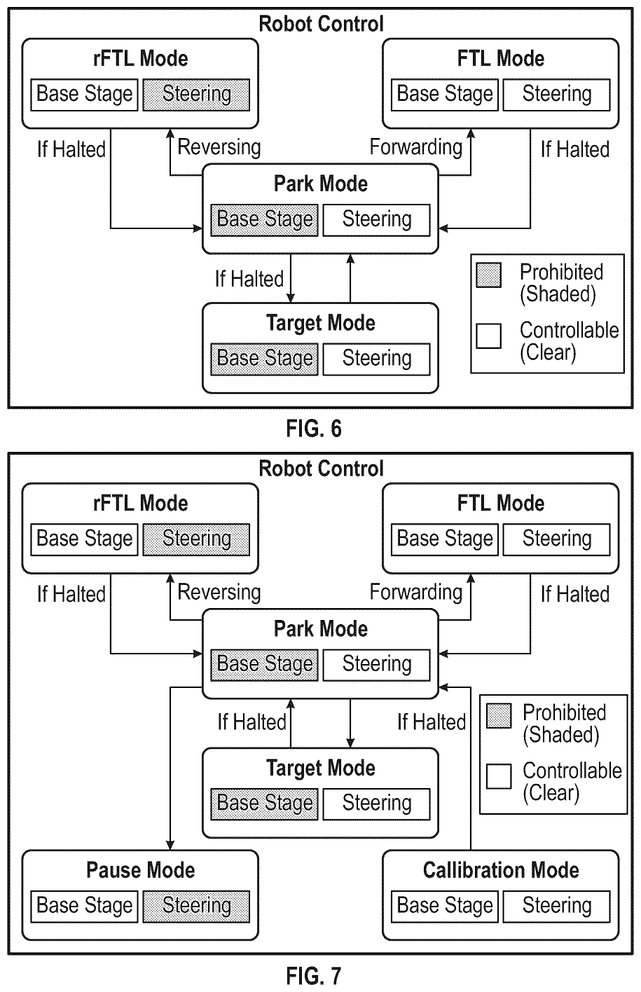

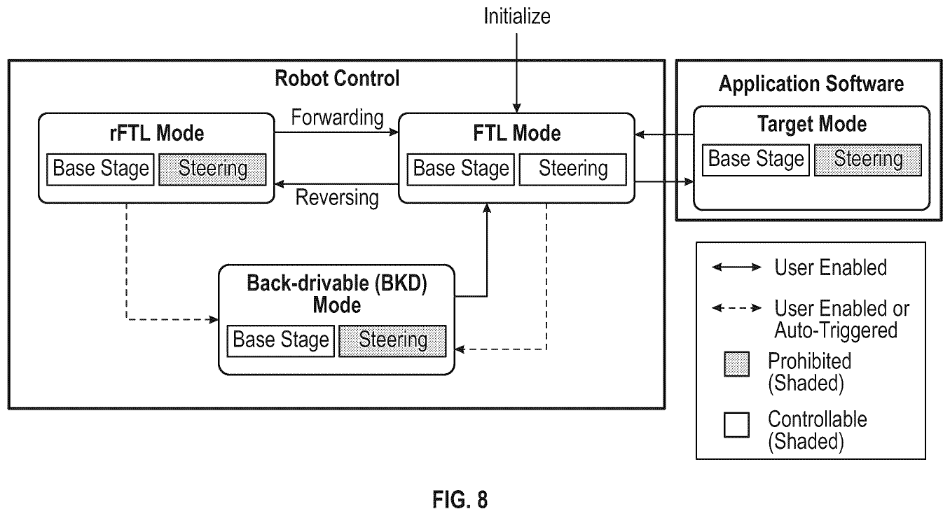

In the clinical workflow, this targeting mode is necessary when the catheter 100 reaches the location close to the target. To include this targeting mode, the park mode can serve as a hub for three driving modes (FIG. 6). As the example design 1 in embodiment 1, FTL mode will be activated with the insertion (forwarding) command, where both base stage 18 and steering tip 172 are permitted under user control. Also, rFTL mode will be activated with removal (reversing) command.

The targeting mode can be activated with the dedicated mode switch button only when the system is in the park mode, i.e., the system does engage/enact have both insertion and removal commands. Moreover, in park mode, the system will allow the operator to control the tip 172 without moving the base stage 18 forward or backward. With this mode switching, the system can reduce human-factor error in accidentally enacting targeting mode during insertion and removal, while the system still provides the intuitive mode selection for the insertion and removal steps.

As a further embodiment, the system can have an additional condition to activate or deactivate the targeting mode from/to the park mode. For example, the system includes a threshold value of a distance between the catheter tip 172 and the target lesion, which the operator can define in the planning step with the navigation system. When the catheter 100 reaches the target lesion within the threshold distance (ex. 25 mm), the system allows the operator to use the targeting mode. With this design, the system further reduces human-factor error.

Moreover, when the system exits targeting mode, the system can include an additional maneuver for confirmation of the catheter 100 shape. The system can provide a “shape-homing” function to the operator, which is to return the catheter 100 shape when the system enters targeting mode from the park mode, and asks the operator to execute this shape-homing function before allowing the operator to exit the targeting mode to the park mode. After the shape-homing function is used, the system allow the operator to exit from the targeting mode when the dedicated mode switch button is pressed. With this design, the system can maintain the continuity of the catheter shape for the FTL and rFTL mode after targeting mode.

### Example Design 2—Current Design for Lung Application With Further Peripheral Motion Modes

This example design includes further motion modes from the example design 1 in this embodiment. Besides the previous three motion modes, this design example includes a calibration mode and a pause mode (FIG. 7). With both motion modes in association with the park mode.

In the calibration mode, the system confirms catheter 100 performance integrity with the base stage 18 in both a disabled or enabled status. Also, in the calibration mode, the system will establish sensor 170 origin for the catheter bending sections 102, 104 and 106, and the base stage 18. After the calibration mode, the system will activate the park mode. From the park mode, the operator cannot activate the calibration mode freely.

In the pause mode, the system freezes the catheter 100 poses and the insertion position. The operator can use this mode to maintain the current catheter 100 poses and the insertion position when the operator would like to release their hands from the joystick operational console 15, or when the operator would like to halt the procedure for some reasons.

The transition from the calibration mode to the park mode can be an event driven activation with the navigation software. After the system confirms the integrity of the catheter 100 and the base stage 18 and the sensor 170 origins, the navigation software can ask the operator if the system can move to the navigation procedure. With the operator's approval, the system can activate park mode.

The transition between the pause mode and the park mode can be implemented with the dedicated mode switch button a foot switch, or the like.

The advantages of the subject innovation is that by mapping the different motion modes based on the insertion and removal commands, the system can provide intuitive robotic control to use the appropriate motion modes for the procedure among the multiple motion modes. The proposed configuration reduces the reliance upon operators' judgement of the mode selection when compared to the conventional mode switch button, which in turn would reduce human-factor error in selecting the wrong motion mode.

Further advantages teach us that by mapping the other motion modes from the motion modes mapped to the insertion and removal commands to the park mode, the system can provide various combination(s) of the other modes with the motion modes mapped to the insertion and removal commands with intuitive robotic control. In this configuration, the system can eliminate the risk of using these additional motion modes during the insertion and removal steps, which reduced the mental burden otherwise borne to the operator.

In conclusion, the subject innovation provides, amongst others, a number of advantages including: An rFTL control algorithm automatically controls the tip 172 section, while the user cannot. To allow tip 172 control after reversing, the system enters Park mode when rFTL is halted, and eliminates the requirement of short range movement; Back-drivable mode can now exit into Park mode, through which transitions into all other options of driving modes, including reverse rFTL, are now available; Forward FTL mode only accepts command on tip 172 or distal bending section 102, but not middle bending section 104 or proximal bending section 106, while target mode is recognizable within DSP robot control software as a distinguishable driving mode to accept commands for both tip 172 or distal bending section 102 and middle bending section 104.

While the present disclosure has been described with reference to exemplary embodiments, it is to be understood that the present disclosure is not limited to the exemplary embodiments described.

1. A robotic apparatus comprising:
a continuum robot including a first bending section which is bent by at least a first wire;
a driver that drives the wire;
a base affixed to the driver and capable of moving the continuum robot in one axis; and
an operational console that controls a movement of the driver and a movement of the base, based on an input,
wherein, the operational console allocates a different motion mode for the robotic apparatus based on an advancing motion or a retracting motion of the robotic apparatus;
wherein the motion modes are mappings between the input and the movement of the driver and movement of the base.

2. The apparatus of claim 1, wherein the continuum robot further comprises a second bending section proximal to the first bending section, which is bent by at least a second wire driven by the driver.

3. The apparatus of claim 1, further comprising a park mode based on a stopping of advance or retraction motion of the robotic apparatus, wherein the mapping between input and movement of the base is suspended and the mapping between input and movement of the driver is allowed.

4. The apparatus of claim 3, wherein, the operational console allocates:
a FTL movement mode based on an advancing motion;
a rFTL movement mode or a relax movement mode based on a retracting motion; and
the park mode based on a stopping of advance or retraction motion.

5. The apparatus of claim 1, wherein park mode is used as a transition from any other modes, including but limited to: a FTL mode, a rFTL mode, a Target mode, a Pause mode, and a relax mode.

6. The apparatus of claim 1, further comprising a sensor to detect a status of the driving wire, wherein the status is a force or a position.

7. The apparatus of claim 1, further comprising an insertion sensor in communication with the continuum robot, to detect the advancing motion, retracting motion or static motion of the continuum robot in a cavity.

8. The apparatus of claim 1, further comprising a pause mode that suspends the operational console from moving the base and the at least first wire.

9. The apparatus of claim 1, further comprising a relax mode that releases a force on the at least first wire.

10. A method for controlling a robotic apparatus:
the robotic apparatus comprising:
a continuum robot having a first bending section which is bent by at least a first wire;
a driver that drives the wire;
a base affixed to the driver and capable of moving the continuum robot in one axis; and
an operational console that controls a movement of the driver and a movement of the base, based on an input,
wherein the method comprises:
receiving an input from a user or from a sensor to the operational console;
sending a signal from the operational console to the driver and/or to the base, wherein, the operational console allocates a different motion mode for the robotic apparatus based on: an advancing motion or a retracting motion of the robotic apparatus, and the signals sent from the operation console,
wherein the motion modes are mappings between input and the movement of the driver and movement of the base;
advancing or retracting the base based on a signal from the operational console;
advancing or retracting the robotic apparatus based on a signal from the operational console.

11. The method of claim 10, wherein the continuum robot further comprises a second bending section proximal to the first bending section, which is bent by at least a second wire driven by the driver.

12. The method of claim 10, further comprising a park mode based on a stopping of the advancing motion or retraction motion of the robotic apparatus, wherein the mapping between input and movement of the base is suspended and the mapping between input and movement of the driver is allowed.

13. The method of claim 12 wherein, the operational console allocates:
a FTL movement mode based on the advancing motion;
a rFTL movement mode or a relax movement mode based on the retracting motion; and
the park mode based on a stopping of the advancing motion or the retraction motion.

14. The method of claim 10, wherein park mode is used as a transition from any other modes, including but limited to: a FTL mode, a rFTL mode, a Target mode, a Pause mode, and a relax mode.

15. The method of claim 10, further comprising a sensor to detect a status of the driving wire, wherein the status is a force or a position.

16. The method of claim 10, further comprising an insertion sensor in communication with the continuum robot, to detect the advancing motion, retracting motion or static motion of the continuum robot in a cavity.

17. The method of claim 10, further comprising a pause mode that suspends the operational console from moving the base and the at least first wire.

18. The method of claim 10, further comprising a relax mode that releases a force on the at least first wire.
